(19) United States
(12) Patent Application Publication
(10) Pub. No.: US 2008/0287782 A1
(43) Pub. Date: Nov. 20, 2008

(54) Radiation Therapy Marking Pen and Method

(76) Inventors: Maeghan Traboulsi, Boston, MA (US); Samuel Cotte, Shelton, CT (US); Mark Broyles, Plainville, CT (US)

Correspondence Address: MCCARTER & ENGLISH LLP;CITYPLACE I, 185 ASYLUM STREET, HARTFORD, CT, 06103, US

(21) Appl. No.: 12/055,269

(22) Filed: Mar. 25, 2008

Related U.S. Application Data
(60) Provisional application No. 60/920,051, filed on Mar. 26, 2007.

(51) Int. Cl. A61N 5/00

(52) U.S. Cl. 600/426

(57) ABSTRACT

A marking device for use in radiation therapy includes a longitudinal axis and a plurality of individual marking members. Each of the plurality of marking members includes a marking tip and a supply of marking fluid, such as an ink. disposed in each marking member. Each of the plurality of marking members is removably positioned relative to the others in succession along the longitudinal axis. In some embodiments, the marking device includes a hollow elongated body having a first end and a second marking end defining an opening, and the plurality of individual marking members are arranged in succession along the hollow body. In other embodiments, each marking member includes a body defining a recess adapted to receive a marking tip of another marking member, and each body includes a connector adapted to releasably interconnect adjacent marking members.

### CROSS-REFERENCE TO PRIORITY APPLICATION

This patent application claims priority on co-pending U.S. Provisional Patent Application Ser. No. 60/920,051, filed Mar. 26, 2007, entitled “Marking Pen”, which is hereby expressly incorporated by reference in its entirety as part of the present disclosure.

### FIELD OF THE INVENTION

The present invention relates to marking devices, and more particularly, to marking devices for radiation therapy having a plurality of single-use tips.

### BACKGROUND

A typical step in radiation therapy is the preparatory step of marking a patient's skin to show a radiologist or technician where to aim the radiation or to provide other information useful for applying the radiation. Marks may be made on the skin to identify iso-centers, three point set-ups, field borders, match lines, etc. A marking pen is used to make colored lines and other marks on the areas of the patient that are to be subjected to the radiation therapy. Typical marking pens used to make these marks are permanent markers, such as Sharpies, and paint pens.

Many practitioners tend to re-use marking pens on multiple patients, since marking pens are generally capable of marking many times over. It is thought that the vast majority of radiation therapy centers and other facilities that provide radiation therapy services do not use a fresh marking pen for each patient. Consequently, a single marking pen may be used on many patients. It is thought that the average marker used for radiation therapy is used on around 85 patients.

The re-use of these marking pens on numerous patients is unsanitary and potentially dangerous. The tip of a marking pen can potentially retain and/or absorb various particles, oils, dead skin, or other matter present on a patient's skin, as well as bacteria or other microbes that may be on the patient's skin or in the surrounding environment, which can then be transferred to another patient upon re-use of the marking pen.

It is an object of the present invention to overcome one or more of the above-described drawbacks and/or disadvantages of the prior art.

### SUMMARY OF THE INVENTION

The present invention is directed to a marking device comprising en elongated axis and a plurality of individual marking members. Each of the plurality of marking members includes a marking tip and a supply of marking fluid such as an ink disposed in each marking member, and is removably positioned relative to the others in succession along the elongated axis.

In accordance with one aspect, the marking device is for radiation therapy, and comprises an elongated axis and a plurality of individual marking members. Each of the plurality of individual marking members includes a marking tip and a supply of marking fluid disposed on the respective marking member for marking an area of a patient's skin and providing information regarding the application of radiation to the patient. One of the marking tips is located in an exposed operative position for marking an area of a patient's skin. Each of a plurality of the individual marking members includes a recess receiving therein the marking tip of a respective trailing marking member, and the trailing tips are maintained in a fresh and substantially sealed condition until ready for use. Each of a plurality of marking members is releasably interconnected to a respective trailing marking member to allow removal of the marking member and, in turn, expose the fresh tip of a respective trailing marking member for use.

In one embodiment, the marking device includes a hollow elongated body having a first or proximal end, a second or distal marking end, an opening at the second marking end, and a plurality of individual marking members arranged in succession along the hollow body. In another embodiment, each marking member includes a body having a recess adapted to receive a marking tip of another marking member, and the body includes a connector adapted to releasably connect the marking member to another marking member.

In accordance with another aspect, the present invention is directed to a marking device for radiation therapy. The marking device comprises a plurality of first means for marking an area of a patient's skin and providing information regarding the application of radiation to the patient. A plurality of second means are provided for releasably coupling the plurality of first means in succession, exposing a leading one of the first means in an exposed operative position for marking an area of a patient's skin, and for maintaining at least one trailing first means in a substantially sealed and fresh condition until ready for use. The device further includes third means for at least one of releasing and ejecting the respective first means located in the exposed operative position and, in turn, exposing a respective trailing first means in a fresh condition for use.

Another aspect of the present invention is directed to a method a method of marking a patient for radiation therapy comprising the following steps:

(i) providing a marking device including a plurality of marking members coupled to one another in succession, wherein each marking member includes a marking tip and a supply of marking fluid on or in fluid communication with the tip, and the tip of one of the plurality of marking members is located in an exposed operative position for marking a patient;

(ii) ejecting the marking member in the exposed operative position from the plurality of marking members coupled to one another in succession and, in turn, locating a fresh marking tip of a successive marking member in the exposed operative position for marking a patient;

(iii) marking an area of a patient's skin with the tip of the fresh marking member in the exposed operative position to provide information regarding the application of radiation to the patient; and

(iv) repeating steps (ii) and (iii) for another patient.

One advantage of the marking devices and methods of the present invention is that they can provide a user with a fresh, clean marking tip for use with each patient without the need to replace an entire marking device. Because a practitioner utilizing the marking devices of the present invention can easily replace a used marking tip with a new marking tip without having to replace the entire marking device, there is neither the need nor the temptation to re-use a single marking tip on multiple patients. Therefore, the present invention can greatly increase the safety and/or cleanliness of procedures in connection with which it is employed, such as radiation therapy.

Other objects and advantages of the present invention and/or of the currently preferred embodiments thereof will become readily apparent in view of the following detailed description of currently preferred embodiments and accompanying drawings.

### BRIEF DESCRIPTION OF THE DRAWINGS

FIG. 1A is a side elevational view of a first embodiment of a marking device of the present invention;

FIG. 1B is a partial cross-sectional view of the marking device of FIG. 1A;

FIG. 1C is a perspective view of the marking device of FIG. 1A showing the ejection of a used marking member therefrom and replacement of the used marking member with a successive fresh marking member;

FIG. 1D is a partial, perspective, partial cross-sectional view of the marking device of FIG. 1A showing the releasable interconnection of a plurality of marking members within the elongated body of the device;

FIG. 2 is a partial, perspective view of a marking end of the marking device of FIG. 1A;

FIG. 3 is a partial, perspective view of a proximal end of the marking device of FIG. 1A;

FIG. 4 is a partial, side perspective view of another embodiment of the marking device of the present invention;

FIGS. 5A, 5B and 5C are somewhat schematic, partial, cross sectional views of the marking device of FIG. 4 illustrating the operation of the marking device;

FIG. 6 is a somewhat schematic, partial cross-sectional view of another embodiment of a marking device of the present invention;

FIG. 7 is a partial, enlarged, cross-section view of the marking end of the marking device of FIG. 6;

FIG. 8 is a side view of an actuator of the marking device of FIG. 6;

FIG. 9 is a side view of a marking tip used in conjunction with the marking device of FIG. 6;

FIG. 10 is a somewhat schematic, cross-sectional view of a plurality of marking members of another embodiment of a marking device of the present invention;

FIG. 11 is a partial, side perspective view of the marking device of FIG. 10;

FIG. 12 is a perspective view of another embodiment of a marking device of the present invention;

FIGS. 13A, 13B and 13C are cross-sectional, top and bottom plan views, respectively, of another embodiment of a marking device of the present invention;

FIGS. 14A and 14B are enlarged, partial cross-sectional views of a portion of the marking device of FIGS. 13A, 13B and 13C.

### DETAILED DESCRIPTION OF THE CURRENTLY PREFERRED EMBODIMENTS

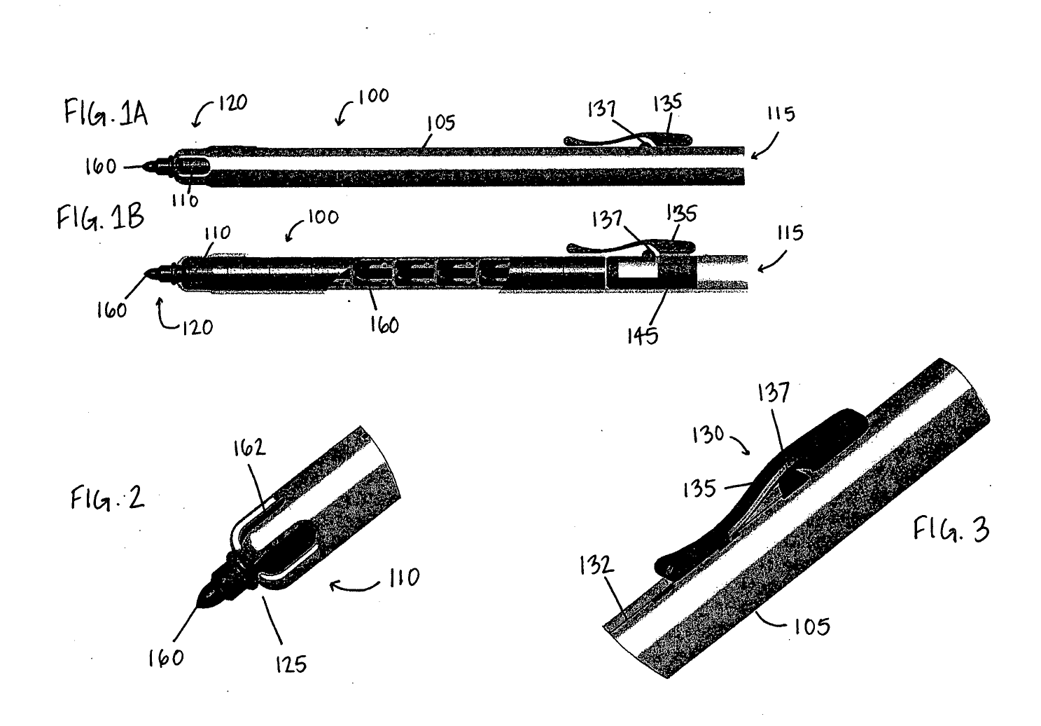

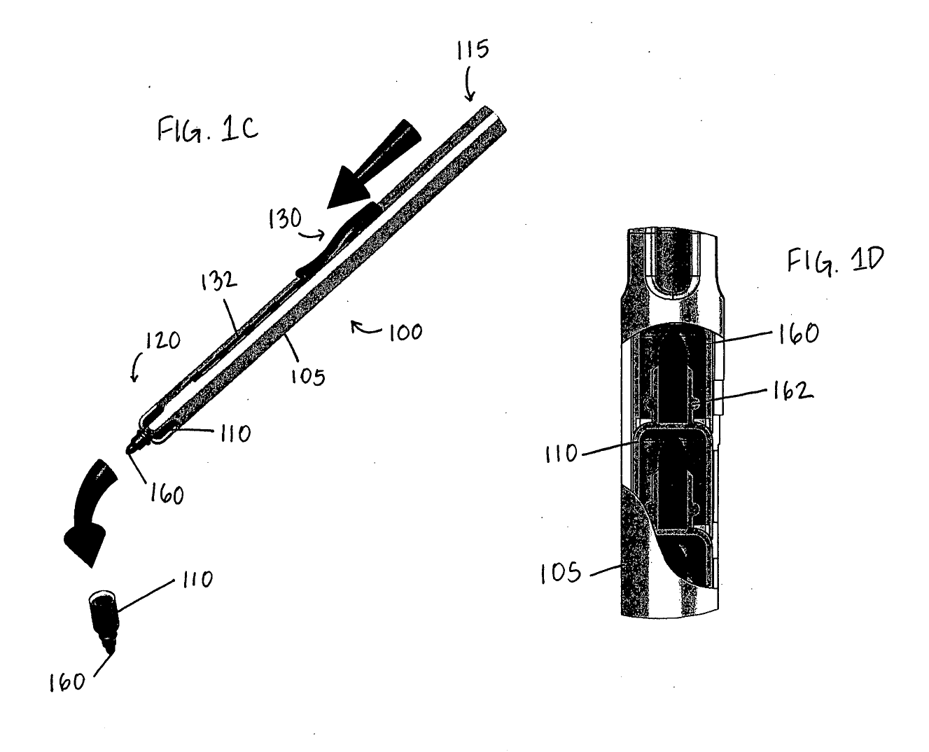

In FIGS. 1A-1D, 2 and 3, a first embodiment of a marking device of the present invention is indicated generally by the reference numeral 100. In this embodiment, the marking device 100 is in a form similar to a writing pen, pencil or marker, and is hereinafter referred to as a “marking pen.” However, the marking pen 100 may take any suitable form conducive to marking a surface that is currently known or used, or that may later become known or used, for this purpose.

The marking pen 100 includes a hollow body or shaft 105 in which a plurality of marking members 110 are disposed. The hollow shaft 105 has a first or proximal end 115 and a second or distal marking end 120. In the embodiment shown in FIGS. 1A-1D, 2 and 3, the hollow shaft 105 defines a circular cross-section; however, any suitable cross sectional shape may be utilized in the marking devices of the present invention. The plurality of marking members 110 are arranged in succession along a length of the hollow shaft 105, and each marking member 110 includes a supply of marking fluid, such as ink, therein. As shown in FIGS. 1A, 1B, 1C and 2, one of the marking members 110 extends through an opening 125 at the distal end of the hollow shaft 105, and is thus exposed to an exterior of the marking pen 100.

The exposed portion 160 of each marking member 110 is known as marking tip, and is used to mark a surface, such as a patient's skin prior to radiation therapy. Each marking tip 160 may take the form of a felt tip or other porous material, and preferably has a marking fluid, such as ink, impregnated therein. In the illustrated embodiment, each marking member 110 is self-contained, in that it has its own supply of marking fluid disposed therein. A marking member 110 may form an individual cartridge including a marking tip 160 and a body 162 for receiving the marking tip therein. For example, as shown in FIGS. 1A, 1B, 1C and 2, each marking member 110 includes a porous marking tip 160 impregnated with ink, and a body 162 to which the respective marking tip 160 is mounted. In other embodiments, each marking member 110 may include a reservoir disposed within its body 162 and in fluid communication with its respective marking tip 160.

As shown in FIG. 1C, after a marking is completed, pressure may be exerted on the stack of successive marking members 110 in the direction of the marking end 120, causing the exposed marking member 110 to progress through the distal opening 125 and be expelled from the marking pen 100. At the same time, the marking member 110 immediately following the expelled marking member 110 in succession is advanced partially through the opening 125, so that at least its respective marking tip 160 extends through the opening 125. The newly exposed marking tip 160 may be used for a new marking task, such as marking a second patient for radiation therapy.

Preferably, each marking member 110 is proximate to or abuts another marking member 110 along an elongated axis of the marking pen 100. When pressure is exerted on the marking member 110 closest to the proximal end 115, all of the marking members 110 in succession advance toward the opposite marking end 120.

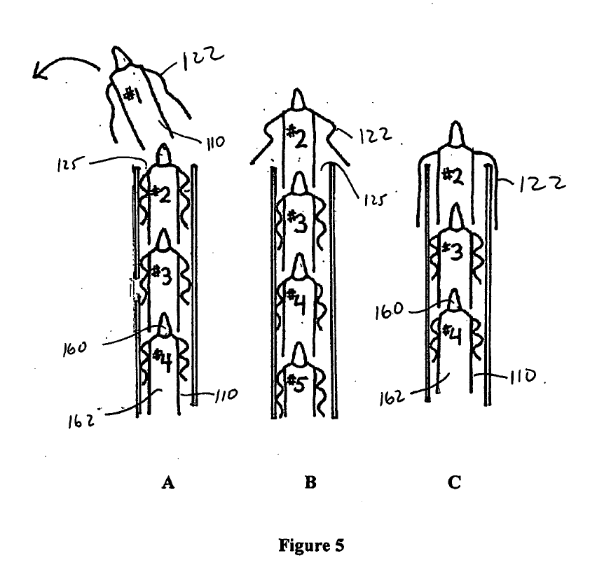

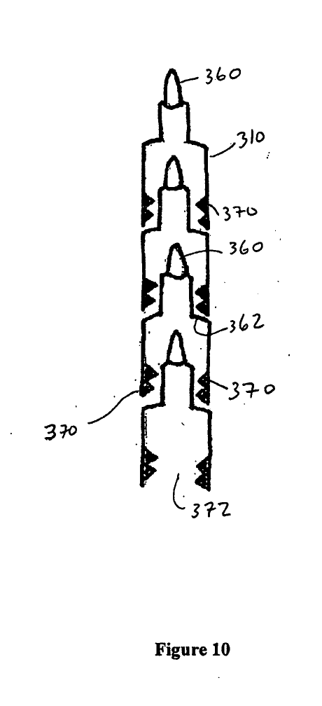

In certain preferred embodiments of the present invention, each body 162 includes a recess in which a marking tip 160 of another marking member 110 is disposed. In this way, the body 162 of a preceding marking member 110 abuts the body 162 of a following marking member 110, and fully encloses the marking tip 160 of the following marking member 110. Accordingly, when the marking members 110 are positioned together and abut one another, each marking tip 160, with the exception of the leading marking tip 160 that is exposed for use, is protected from the surrounding environment, so that contamination or drying out can be substantially avoided. The marking members of this embodiment may be disposed in a hollow body or shaft 105, as shown in FIGS. 1A, 1B, 1D, or as described further below in connection with FIGS. 5, 6, 7, 13A, 13C, 14A and 14B, or may be stacked and connected directly together in a substantially rigid, self-contained form, as described further below in connection with FIGS. 10-12, thereby avoiding the need for a hollow shaft or body.

In one embodiment, the marking pen 100 includes an airtight seal that may be placed over the marking end 120 to prevent the ink or other marking fluid in the exposed marking tip 160 from drying out. This seal may be in the form of a rigid cap that is fit over the marking end 120, a plastic or other airtight film placed over the exposed marking tip 160 and removably adhered to the hollow shaft 105, or any other device that is currently known or used, or that later may become known or used for this purpose.

In one embodiment, the marking pen 100 also includes an actuator 130 for exerting pressure on the plurality of marking members 110 to expel a used marking member 110 and expose a fresh marking tip 160 on a new marking member 110. In the embodiment shown in FIGS. 1A, 1B, 1C and 3, the actuator 130 is in the form of a sliding clip or other member that may be advanced toward the marking end 120. The actuator 130 also includes an interior portion 145 that extends into the interior of the hollow shaft 105 when the actuator 130 is positioned on the hollow shaft 105. In the embodiment shown in FIGS. 1-3, the interior portion 145 is in the form of a plunger for engaging and moving the marking members with the actuator.

As shown in FIG. 1C, the actuator 130 is engaged by manually pressing the exterior portion 135 to move the actuator 130 toward the second marking end 120, thereby causing the interior portion 145 to exert pressure on the plurality of marking members 110 and, in turn, expel a currently exposed marking tip 160 and expose the marking tip 160 of a trailing marking member 110 for use. The hollow shaft 105 may define a slit 132 or elongated opening formed along a selected length of the hollow shaft 105 to allow the interior portion 145 to advance in an axial direction along the interior of the hollow shaft 105 with the exterior portion 135 of the actuator.

As shown in FIGS. 1A, 1B, 1C and 3, a biasing member 137 is attached to the actuator 130. The biasing member 137 may be integrally formed with the interior portion 145 and operates in conjunction with periodically placed enlarged portions of the slit 132. The biasing member 137 holds the actuator 130 in place at the location of each enlarged portion of the slit 132, so that the actuator 130 can be advanced incrementally toward the marking end 120. The added resistance helps to prevent the actuator 130 from being pushed too far and accidentally expelling more than one marking member 110 at a time.

Although the actuator 130 is described as being actuated by pressing or advancing the actuator portions toward the marking end 120, the actuator may take various other forms or employ other mechanisms for exerting pressure on or otherwise advancing the plurality of marking tips. Other mechanisms may include a threaded mechanism that is advanced by rotation, a piston, a lever, or any other mechanisms that are currently known or used, or that later become known or used, for this purpose.

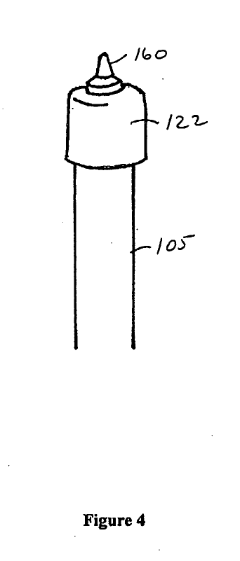

Another embodiment of marking pen 100 is shown in FIGS. 4, 5A, 5B and 5C, in which each marking member 110 includes a collapsible and expandable shield cover 122. Each shield cover 122 may be made from a plastic or rubber material, such as silicone, or other material having sufficient flexibility to be collapsed and expanded. The shield cover 122 has a first collapsed position in which the shield cover 122 is collapsed around the bodies 162 of the interior marking members 110, as shown in FIG. 5A. Upon advancement of the marking member 110 sufficiently beyond the opening 125, as shown in FIG. 5B, the shield cover 122 is expandable to a second expanded position in which it forms a shape large enough to cover the opening 125. After the shield cover 122 has fully expanded, the respective marking member 110 may be retracted to a working position, as shown in FIG. 5C, in which the shield cover 122 covers and protects both the opening 125 and the interior of the hollow shaft 105. The shield cover 122 may be attached to the body 162 of each marking member 110, such as with an adhesive, or may be integrally formed with the body 162. Each shield cover 122 also may be both flexible and resilient, so that when a marking member 110 is advanced sufficiently far from the opening 125, the respective shield cover 122 reverts to its original expanded shape.

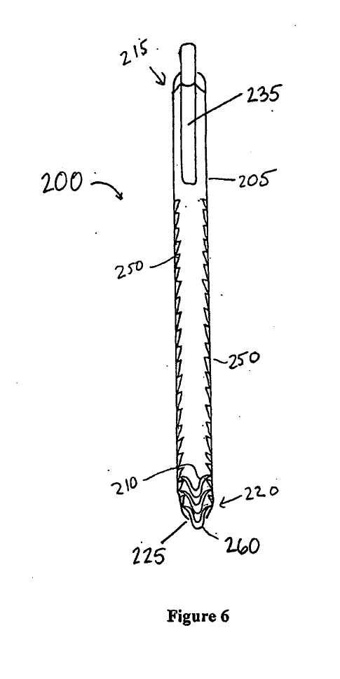

Turning to FIGS. 6 through 9, a marking pen 200 is shown with alternative embodiments of marking members 210 and additional embodiments for securing and advancing the marking members 210. The features of the marking pen 200 are similar to those of the marking pens 100 described above, with the exception of the features hereinafter described, and therefore like reference numerals preceded by the numeral “2” instead of the numeral “1” are used to indicate like elements.

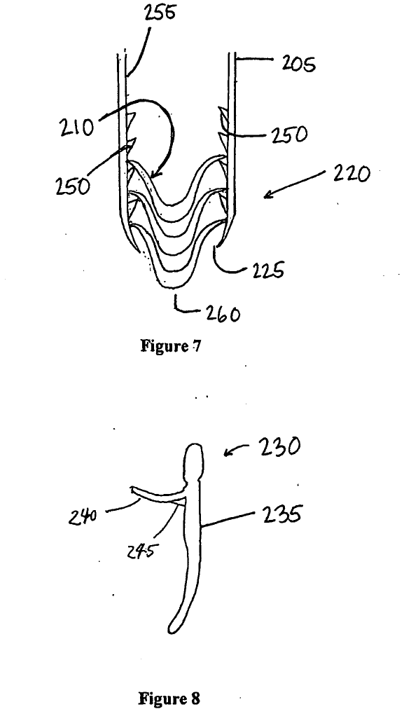

As shown in FIGS. 6 and 7, the marking pen 200 includes mechanisms to hold both the plurality of marking tips 210 and the actuator 230 in place. A plurality of protrusions 250 extend from an interior wall 255 of the hollow body or shaft 205, each creating a ridge or ledge at a different position along the length of hollow shaft 205, upon which either one of the plurality of marking tips 210, or actuator 230, are supported.

The actuator 230 works in conjunction with protrusions 250 to prevent the actuator 230 from moving axially along the hollow shaft 205 when the actuator 230 is not engaged. The actuator 230 includes an exterior portion 235 that is positioned on the exterior of the hollow shaft 205, which may include a clipping portion 240 shaped to hold the actuator 230 on the hollow shaft 205. This aspect also further secures the marking tips 210 in place, as the actuator 230 also acts as a stopper, preventing axial movement of the marking tips when the actuator 230 is not engaged. The interior portion 245, which exerts pressure on the plurality of marking tips 210 when the actuator 230 is engaged, is held in place and otherwise supported by the protrusions on either side of the interior portion 245.

In one embodiment, the interior portion 245 is a resilient member that flexes when the actuator 230 is engaged, to allow the interior portion 245 to move past the respective protrusion(s) 250 toward the marking end 220.

As shown in FIGS. 6, 7 and 9, each marking member 210 preferably includes its own supply of ink or other marking fluid, and is thus self-contained, such that no external supply of ink is necessary. In one embodiment, at least a marking tip 260 of each marking member 210 is made from a porous material that is at least partially impregnated with marking fluid. The porous material may be any suitable material capable of retaining a fluid, such as a foam material, a fabric or a felt material.

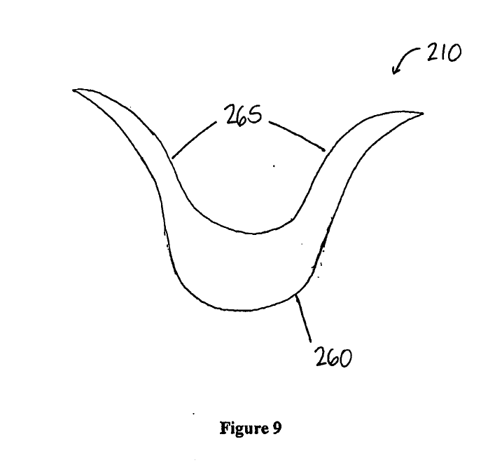

As shown in FIG. 9, each marking member 210 includes a marking tip 260, and a resilient rear body portion 265. Each marking tip 260 is preferably rounded, but may be any suitable shape, such as conical, pointed, cylindrical, etc. The resilient rear portion 265, in the current embodiment, has a rounded shape, but also may be one of many shapes that are currently known or used, or that later become known or used for this purpose. In one embodiment, the resilient rear portion 265 defines a recess having a shape generally designed to accommodate a marking tip 260 of another marking tip 210, so that a plurality of marking tips 260 may be stacked on one another with minimal space therebetween within the hollow shaft 205. This tip shape and spatial arrangement permits the marking pen 200 to accommodate a large number of marking members 210, which in turn increases the number of times the marking pen 200 may be used without replacing or reloading with additional marking members 210.

As also shown in FIG. 9, each resilient rear portion 265 defines a width that is greater than a width of the respective marking tip 260. As is shown in FIG. 7, when the plurality of marking members 210 are loaded into the marking pen 200, the resilient rear portion 265 of a leading exposed marking member 210 is supported against the interior wall 255 of the hollow shaft body 205, while the narrower marking tip 260 extends out through the opening 225.

As shown in FIG. 7, the plurality of protrusions 250 described above also support each marking member 210. Each of the plurality of marking members 210 is supported and held in place by a respective ledge formed by one or more of the plurality of protrusions 250. In particular, the resilient rear portion 265 of each unexposed marking tip 210 is supported by a respective ledge. When pressure is exerted on the plurality of marking members 210, such as via the manually-engageable actuator 230, the resilient rear portion 265 of each marking member 210 flexes or deforms, and each marking member 210 advances to either a ledge closer to the opening 225, or to the opening 225 itself.

As shown in FIG. 7, each unexposed marking member 210 is supported by at least one protrusion 250. Alternatively, each unexposed marking member 210 may be supported by two or more protrusions 250 positioned at various locations on the interior portion 255 in a plane at least substantially perpendicular to the elongated axis of the hollow body 205. In another embodiment, each protrusion 250 forms an at least partially annular ring.

As shown in FIGS. 6 and 7, the marking end 220 defines an opening 225. At least a portion of the marking end 220 extending from the opening 225 is made from a flexible and resilient material that causes the opening 225 to temporarily enlarge when pressure is exerted on the plurality of marking members 210 to, in turn, allow an exposed marking member 210 to be expelled from the marking pen 200.

The marking end 220 defines a first position and a second position. In the first position, the opening 225 has a width that is at least as great as the width of marking portion 260, but is less than the width of the resilient rear portion 265. In this first position, the opening 225 allows the marking tip 260 of a marking member 210 to be exposed, while the interior wall 255 supports the resilient rear portion 265 and thereby prevents the exposed marking member 210 from slipping through the opening 225. In the second position, which occurs as pressure is exerted on the plurality of marking members 210 to expel the exposed marking member 210, the walls around the opening 225 are forced to temporarily deform outwardly to enlarge the opening 225 and, in turn, allow the exposed marking member 210 to progress through the opening 225. After the exposed marking member 210 is forced through the opening 225, the opening 225 returns to its previous size and shape, and provides support for the newly exposed marking tip 210.

In FIGS. 10-12, another embodiment of a marking pen of the present invention is indicated generally by the reference numeral 300. The marking pen 300 includes a plurality of marking members 310 connectable together to form a rigid, self-contained marking pen without a hollow body or shaft for receiving therein the marking members as described above. The features of the marking pen 300 are similar to those of the marking pens 100 and 200 described above, with the exception of the features hereinafter described, and therefore like reference numerals preceded by the numeral “3” instead of the numerals “1” or “2” are used to indicate like elements.

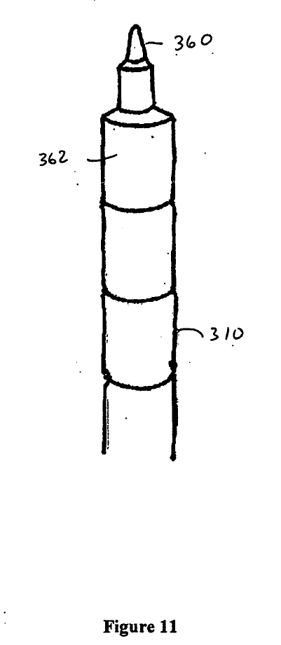

Each marking member 310 includes a marking tip 360, a marking body 362, and a connector or fastener 370. Each marking body 362 also includes a recess 372 adapted to receive a marking tip 360 of another marking member. In the embodiment shown in FIGS. 10-11, each connector 370 includes a feature on the respective body 362 that affects a screw or twist connection to lock successive marking members together. As also shown, when the marking members 310 are connected, each marking tip 360, with the exception of the leading exposed marking tip 360, is disposed within a preceding recess 372 and is thus protected from the surrounding environment. Each connector 370 may include one or more grooves or threads on the interior of the respective body 362 and/or one or more grooves or threads on the exterior of the respective body 362 to connectively cooperate with the interior threads of another marking member.

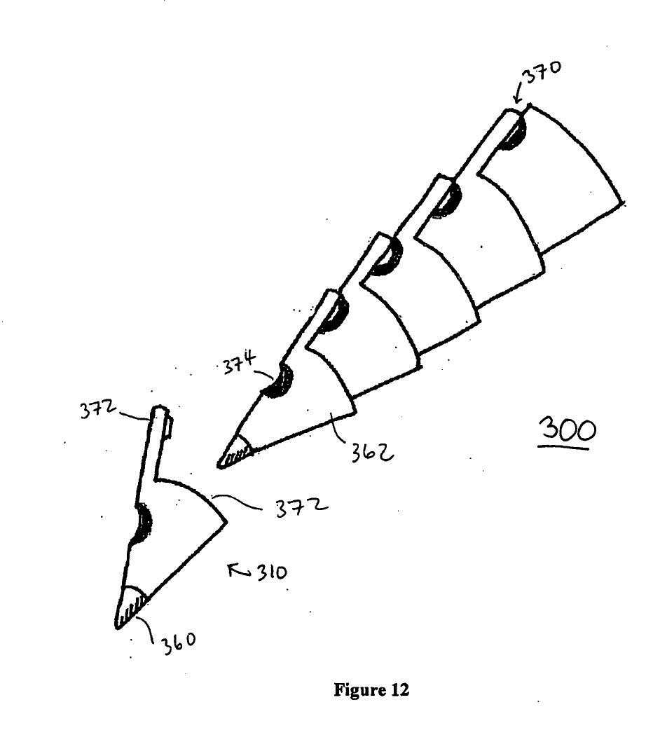

FIG. 12 shows an alternative embodiment of the marking members 310 in which a plurality of the marking members are releasably connectable to form a rigid, self-contained marking pen without an elongated body or shaft for receiving and supporting the marking members. In this embodiment, each marking member 310 defines a substantially conical external shape, and a substantially conical recess 372 into which the marking tip 360 of a respective trailing marking member 310 is received to interconnect the marking members. Also in this embodiment, each connector 370 includes a tab 372 extending from the respective body 362, and a locking recess 374 located on each body 362. Each tab 372 is configured to be received in and removably fastened to a respective locking recess 374. Each tab 372 may be a snap tab or clip that forms a snap connection with a respective locking recess 374, or that may form an interference or press fit with a respective locking recess 374. One advantage of this configuration is that a user can simply flick the tab of the leading marking member 310 to, in turn, allow the leading marking member 310 to fall away from marking pen 300. Accordingly, the user can remove the used marking member 310 with minimal contact.

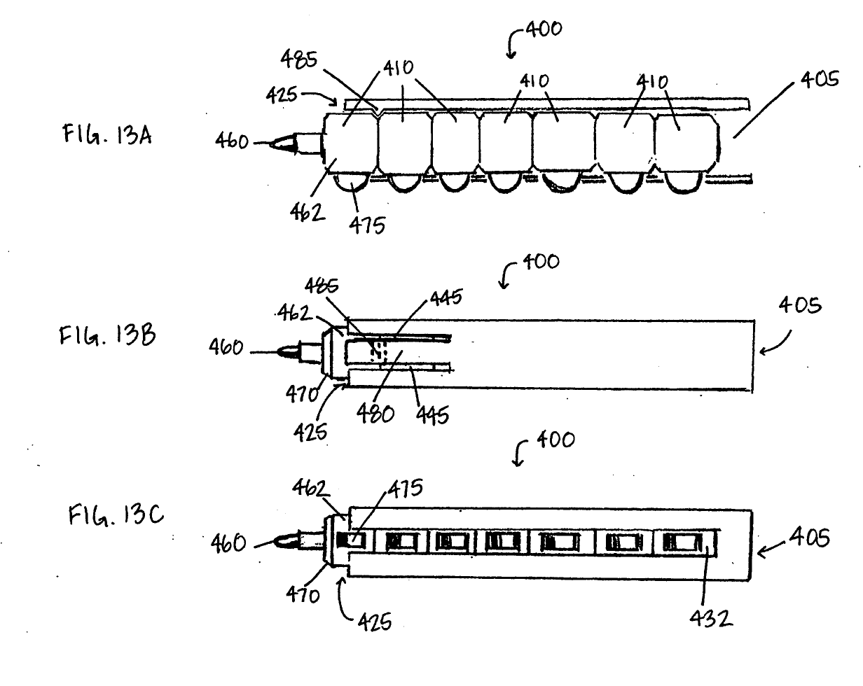

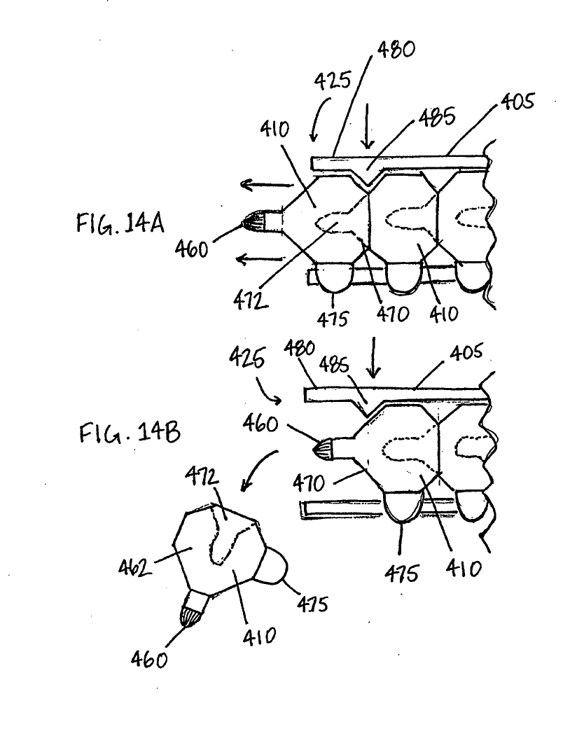

Turning to FIGS. 13A through 13C, 14A and 14B, another embodiment of a marking device of the present invention is indicated generally by the reference numeral 400. The marking pen 400 includes a plurality of segmented marking members 410 within a pen barrel 405. The features of the marking pen 400 are similar to those of the marking pens 100, 200 and 300 described above, with the exception of the features hereinafter described, and therefore like reference numerals preceded by the numeral “4” instead of the numerals “1,” “2,” or “3” are used to indicate like elements.

As shown in FIGS. 13A through 13C, the marking pen 400 includes a plurality of segmented marking members 410 joined in series and disposed within a pen barrel 405. Each of the plurality of marking members 410 includes a marking tip 460, a marking body 462, a connector or fastener 470, a recess 472 adapted to receive a marking tip 460 of another marking member 410, and an advancement tab 475. As shown in FIG. 13A, the marking members 410 are releasably conjoined along an elongated axis of the marking pen 400. Each marking member 410 is self-contained, in that it has its own supply of marking fluid disposed therein.

As shown in FIGS. 13A and 13B, a barrel extension 480 is disposed near the marking end 420 on a portion of the pen barrel 405. The barrel extension 480 is a slender portion of the pen barrel 405 formed by cutting longitudinal slits 445 into the pen barrel 405. A pointed protrusion 485 is located on the interior of the barrel extension 480, within the pen barrel 405, and during operation is positioned at the junction of the leading marking member 410 and the connector or fastener 470 of the trailing marking member 410. Because each of the plurality of marking members 410 is releasably conjoined, the pointed protrusion 485 frictionally maintains the various marking members 410 in their appropriate longitudinal positions.

As shown in FIG. 13C, the pen barrel 405 is hollow and features a longitudinal tab channel 432, within which advancement the tabs 475 of the plurality of marking members 410 are aligned. The tab channel 432 permits a user to contact and manipulate each of the advancement tabs 475 from the exterior of the pen barrel 405. At the opening 425 of the marking end 420, the marking tip 460 of the leading marking member 410 is exposed for marking a surface, such as a patient's skin in preparation for radiation therapy.

After a patient marking as been completed, the leading marking member 410 may be removed from the marking pen 400 by applying radially inward pressure, such as with a thumb or forefinger, to the barrel extension 480 near the opening 425, as shown in FIG. 14A. If sufficient pressure is applied to the exterior of the pen barrel 405, the pointed protrusion 485 opens the connection between the recess 472 of the leading marking member 410 and the connector or fastener 470 of the trailing marking member 410. As shown in FIG. 14B, the leading marking member 410 may then be removed by physically agitating or shaking the marking pen 400, pushing its advancement tab 475 toward the opening 425 and out of the pen barrel 405, or any other suitable method or technique. Any marking members 410 remaining within the pen barrel 405 may then be advanced to the appropriate longitudinal position for operation by pushing their advancement tabs 475 toward the opening 425. The barrel extension 480 flexibly permits the next marking member 410 to slide into the appropriate longitudinal position for operation, and then snaps into place between the next marking member 410 and any following marking member 410.

The binding relationship between the leading marking member 410 and the trailing marking member 410 is shown in FIGS. 14A and 14B. Although the marking tip 460 of the leading marking member 410 is exposed, the marking tip 460 of the trailing marking member 410 is releasably sealed airtight, or substantially airtight, within the recess 472 in the body 462 of the respective leading marking member 410, to prevent the ink or other marking fluid in the trailing marking tip 460 from drying out, and to otherwise protect the marking tab 460 from contamination or damage. In the embodiment shown in FIG. 13A through 13C, 14A and 14B, the releasable seal between connected marking members 410 is a taper lock formed by mating the connector or fastener 470 of the trailing marking member 410, which is substantially frustoconical in shape, with the recess 472 of the leading marking member, which is adapted to receive the connector or fastener 470 of the trailing marking member 410. The mating of the connector or fastener 470 of the trailing marking member 410 and the recess 472 of the leading marking member 410 forms a substantially airtight seal around the marking tip 460 of the trailing marking member 410. However, any other joining arrangement may be utilized to releasably link the marking members 410, such that a substantially airtight seal is maintained about the marking tip 460 of the trailing marking member 410, and relative motion between the marking members 410 is substantially prevented.

The connectors and connecting schemes described above are purely exemplary. As may be recognized by those of ordinary skill in the pertinent art based on the teachings herein, any suitable connector or connecting mechanism may be employed that removably connects or fastens the marking members, including any schemes or mechanisms that are currently known or used, or that may later become known or used, for this purpose.

As discussed above, the marking tips may be made from porous materials such as foam material, fabric material or felt material. However, marking tips may be made from any suitable material capable of retaining fluids, or may be made from a plastic, rubber or other material configured to allow the passage of fluids. In addition, the various components of the marking pens discussed above, including the hollow shaft, marking member bodies and actuator, are preferably made from a plastic material. However, these components may be made from any suitable material that is currently known or used, or that later may become known or used.

The marking pens of the present invention may be advantageously utilized for many purposes or procedures. An exemplary method includes providing a marking device such as marking pen 100 having a hollow elongated body or shaft 105 with a first or proximal end 115 and a distal or marking end 120, and a plurality of individual marking members 110, each having a supply of marking fluid therein, arranged in succession along the hollow shaft 105. The method further includes, after use of an exposed marking member 110, exerting pressure on the marking member 110 nearest the first end 115 to eject the used marking member 110 from marking pen 100 and advance a new tip to an end of the marking pen 100, and marking a surface with the new marking member 110.

An alternative exemplary method may be performed in conjunction with, for example, any of the marking pens 300. This method includes providing a marking device 300 including a plurality of the individual marking members 310 connected in succession and marking an area of a patient's skin with the marking tip 360 of an exposed leading marking member 310. After the patient marking is complete, a connection between the leading marking member 310 and a trailing marking member 310 is released to expose the marking tip 360 of a respective trailing marking member 310. The used leading marking member 310 is then discarded, and an area of a second patient's skin is marked with the marking tip 360 of the newly exposed trailing marking member 310.

The methods and marking devices described herein are suitable for many tasks that require marking a surface, and are particularly suitable for tasks where it is desirable to provide a fresh, unused marking tip for each use. For example, the above devices and methods may be utilized in marking a patient in preparation for radiation therapy, in which an area of a patient's skin is marked to provide information regarding the application of radiation to the patient. These devices and methods have many additional uses, in medical and other applications. For example, these devices and methods may be used in pre-surgical planning, such as for verifying the correct surgical site, planning for plastic surgery, etc.

The present invention is particularly advantageous for radiation therapy or other medical uses, in that a user may use the same marking pen for multiple patients, while providing a fresh, unused marking tip for each patient. Thus, the marking pens described herein help to ensure that each patient is marked with a clean tip, so that particles or microbes are not transferred between patients, while avoiding the inconvenience and waste associated with providing a brand new marking device for each patient.

As may be recognized by those of ordinary skill in the pertinent art based on the teachings herein, numerous changes and modifications can be made to the above-described and other embodiments of the present invention without departing from the scope of the invention as defined in the appended claims. The features of the marking devices described above are only exemplary, and may be changed as desired, or otherwise as required to meet the requirements of a particular application. Accordingly, this detailed description of the currently preferred embodiments is to be taken in an illustrative, as opposed to a limiting sense.

What is claimed is:

1. A marking device for radiation therapy, comprising:
an elongated axis; and
a plurality of individual marking members, wherein each of the plurality of individual marking members includes a marking tip and a supply of marking fluid disposed on the respective marking member for marking an area of a patient's skin and providing information regarding the application of radiation to the patient, wherein one of the marking tips is located in an exposed operative position for marking an area of a patient's skin, each of a plurality of the individual marking members includes a recess receiving therein the marking tip of a respective trailing marking member, the trailing tips are maintained in a fresh and substantially sealed condition until ready for use, and each of a plurality of marking members is releasably interconnected to a respective trailing marking member to allow removal of the marking member and, in turn, expose the fresh tip of a respective trailing marking member for use.

2. A marking device as defined in claim 1, wherein each marking tip is made from a porous material that is at least partially impregnated with the marking fluid.

3. A marking device as defined in claim 3, wherein the porous material is at least partially impregnated with the marking fluid.

4. A marking device as defined in claim 1, wherein each marking member includes a reservoir containing a substantially predetermined quantity of the marking fluid, the reservoir being in fluid communication with the marking tip.

5. A marking device as defined in claim 1, further comprising a hollow elongated body having a first end, a second marking end, and an opening at the second marking end, wherein the plurality of individual marking members are arranged in succession within the hollow elongated body.

6. A marking device as defined in claim 5, wherein one of the plurality of marking tips extends through the opening in the second marking end of the hollow elongated body.

7. A marking device as defined in claim 5, further comprising an actuator having an exterior portion located on an exterior of the hollow elongated body, and an interior portion located in an interior of the hollow elongated body, wherein the actuator is movable along a substantially predetermined length of the hollow elongated body.

8. A marking device as defined in claim 7, wherein the interior portion is a plunger movably received within the hollow elongated body.

9. A marking device as defined in claim 7, wherein the actuator is manually engageable by pressing the exterior portion to move the exterior portion toward the second marking end, thereby causing the interior portion to exert pressure on the plurality of marking members and at least one of eject and release the exposed marking member from the marking device and, in turn, expose a second marking tip through the opening of the marking end.

10. A marking device as defined in claim 5, wherein the marking end is madc from a resilient material and the opening expands from a first position to a second actuated position when pressure is exerted on the plurality of marking members toward the marking end, the second position defines a width greater than the width of the first position, and thereafter returns to the first position in which the marking end supports the newly exposed marking tip.

11. A marking device as defined in claim 7, further comprising a plurality of protrusions extending from an interior of the hollow body, wherein each protrusion supports the interior portion and prevents the interior portion from sliding along the hollow body.

12. A marking device as defined in claim 11, wherein the interior portion is a resilient member that flexes when the actuator is engaged to release the actuator from a respective protrusion and allow the actuator to be moved toward the second marking end.

13. A marking device as defined in claim 11, wherein each marking tip includes a marking portion that is impregnated with the marking fluid, and each marking member includes a resilient portion having a width that is greater than a width of the marking tip.

14. A marking device as defined in claim 13, wherein the plurality of protrusions form a plurality of ledges axially spaced along the hollow body, and each ledge supports a resilient portion of a respective marking tip and holds each marking tip substantially in place.

15. A marking device as defined in claim 14, wherein each marking member, in response to a substantially predetermined force, deforms so as to move each marking member from a first ledge to at least one of (i) a second ledge closer to the marking end, and (ii) to an opening at the marking end.

16. A marking device as defined in claim 5, wherein each marking member includes a tip, a body, and a flexible shield that is in a collapsed state when the marking member is located within the hollow shaft, and is expandable to cover the marking end when the marking tip extends through the opening.

17. A marking device as defined in claim 1, wherein each marking member includes a connector adapted to removably connect the marking member to another marking member.

18. A marking device as defined in claim 5, further comprising an elongated body receiving therein the plurality of marking members in succession along an elongated axis of the body, and wherein the elongated body includes an extension defining a protuberance engageable between successive marking members for maintaining a respective position of the marking members and facilitating release of an exposed marking member.

19. A marking device for radiation therapy, comprising:
a plurality of first means for marking an area of a patient's skin and providing information regarding the application of radiation to the patient;
a plurality of second means for releasably coupling the plurality of first means in succession, exposing a leading one of the first means in an exposed operative position for marking an area of a patient's skin, and for maintaining at least one trailing first means in a substantially sealed and fresh condition until ready for use; and
third means for at least one of releasing and ejecting the respective first means located in the exposed operative position and, in turn, exposing a respective trailing first means in a fresh condition for use.

20. A marking device as defined in claim 23, wherein each first means is a marking member including a porous marking tip impregnated with a marking fluid, and a marking body, each second means is a connector adapted to removably interconnect adjacent marking members, and each third means is a manually engageable member for manually engaging and at least one of releasing and ejecting a respective first means.

21. A method of marking a patient for radiation therapy, comprising the steps of:
(i) providing a marking device including a plurality of marking members coupled to one another in succession, wherein each marking member includes a marking tip and a supply of marking fluid on or in fluid communication with the tip, and the tip of one of the plurality of marking members is located in an exposed operative position for marking a patient;
(ii) ejecting the marking member in the exposed operative position from the plurality of marking members coupled to one another in succession and, in turn, locating a fresh marking tip of a successive marking member in the exposed operative position for marking a patient;
(iii) marking an area of a patient's skin with the tip of the fresh marking member in the exposed operative position to provide information regarding the application of radiation to the patient; and
(iv) repeating steps (ii) and (iii) for another patient.

22. A method as defined in claim 21, further comprising the steps of:
providing a marking device including a hollow elongated body having a first end and a second marking end; and a plurality of individual marking members arranged in succession along the hollow elongated body with one of the marking members located in the exposed operative position at the marking end of the elongated body; and
exerting pressure on at least one marking member spaced from the marking end and, in turn, ejecting a previously used marking member from the marking end of the marking device and advancing a fresh marking member to the exposed operative position at the second marking end of the marking device.

23. A method as defined in claim 21, further comprising the steps of:
providing a plurality of marking members, wherein each marking member includes a body having a recess adapted to receive a marking tip of another marking member, and each body includes a connector adapted to removably connect the respective marking member to another marking member; and
manually releasing the connection between the leading marking member and a respective trailing marking member and, in turn, exposing the marking tip of the trailing marking member into the exposed operative position.
